(19) United States
(12) Patent Application Publication
(10) Pub. No.: US 2026/0144460 A1
(43) Pub. Date: May 28, 2026

(54) Device for Measurement of Cerebral OxyHb-deOxyHb Phase Relationship

(71) Applicant: Brain Check Medical, LLC, Underwood, MN (US)

(72) Inventor: John A. Borgos, Underwood, MN (US)

(73) Assignee: Brain Check Medical, LLC, Underwood, MN (US)

(21) Appl. No.: 19/389,146

(22) Filed: Nov. 14, 2025

Related U.S. Application Data
(60) Provisional application No. 63/833,608, filed on Nov. 25, 2024.

(51) Int. Cl. A61B 5/145; A61B 5/00; A61B 5/021

(52) U.S. Cl. CPC A61B 5/14546; A61B 5/02141; A61B 5/6803; A61B 5/7221; A61B 5/725; A61B 5/7264; A61B 5/7271; A61B 5/742; A61B 5/746; A61B 2560/0214; A61B 2560/0462; A61B 2562/0233; A61B 2562/0247

(57) ABSTRACT

A system and method for measuring phase relationships comprises a controller, a display, a first light source, a second light source and at least two optical detectors. The light sources and detectors are contained in a sensor patch that is part of a patient wearable headset, wherein when the headset is placed on the patient's head and the sensor patch is held in place over the scalp of the patient's forehead. Light signals transmitted by the light sources into the brain of the patient are detected by the optical detectors, analyzed and processed by the controller, and displayed in the form of a calculated phase value result.

### CROSS-REFERENCE TO RELATED CASES

The present application is a utility filing claiming priority to U.S. Provisional Application No. 63/833,608, filed on Nov. 25, 2024, the entire contents of which is incorporated herein by reference.

### BACKGROUND

Near infrared spectroscopy (NIRS) devices are used in commercially available instrumentation for measurement of the concentrations of chromophores of interest in biological tissues. Principles of NIRS measurements in cerebral tissue are well known, and noninvasive devices with light sources and detectors applied to the skin surface are commonly used in clinical practice. In the present disclosure, the most relevant chromophore is hemoglobin (Hb), which dominates the absorption of photons in a useful range of the near infrared (NIR) spectrum (between 600 and 1350 nanometers). The two primary Hb species are oxygenated Hb (OxyHb) and de-oxygenated Hb (deOxyHb). Conveniently, OxyHb absorption is different than deOxyHb at most near infrared (NIR) wavelengths; deOxyHb absorption is greater than OxyHb absorption at wavelengths less than 800 nanometers, and less than OxyHb absorption at wavelengths above 800 nanometers. With judicious selection of the two or more NIR wavelengths, and two or more source-detector separation distances, application of the well-known Beer-Lambert law (BLL) permits measurement of the OxyHb and deOxyHb concentrations in cerebral tissue. Modified versions of the BLL can be used to account for optical pathlength dependence on the NIR wavelength. In the present disclosure, the term “optical wavelength” will be used to refer to a wavelength of light emitted by a NIR light source.

Blood flow in cerebral arteries and tissues is essentially non-constant, varying in both periodic and non-stationary patterns. Periodic variations include those caused by cardiac cycles and respiration cycles, which are mechanically coupled to intra-arterial and intra-thoracic pressure variations. Periodic variations caused by respiration cycles may correspond to the actual respiration rate, but may differ if the respiration rate is very fast, such as 25/minute, or very slow, such as 4/minute.

Periodic variations in cerebral blood flow may also include Mayer waves, which have been reported in intracranial pressure measurements as well as in cerebral artery velocity measurements. The periods of these waves are typically near 10 seconds, and they are often referred to as “low-frequency” (LF) variations. Periods may vary from 5 -15 seconds. Successive wavelengths may differ such that periodicity is not strictly constant. Mayer waves are thought to originate from cerebral and central processes that are, at least to some extent, independent of mechanical effects. In the present disclosure, a waveform period of a physiological parameter variation, which includes OxyHb and deOxyHb for example, and a waveform period in the signal generated by a measurement of a physiological parameter, are represented by the term “signal wavelength.”

In healthy subjects, the LF phase relationship between OxyHb and deOxyHb oscillations has been described as counterphase, meaning that their waveforms are separated by approximately 3.14 radians. This can be seen in the LF waveforms of OxyHb and deOxyHb after bandpass filtering as shown in PRIOR ART FIG. 1a, and the series of lead time (LT) and phase calculations corresponding to the waveforms as shown in PRIOR ART FIG. 1b. The results shown in PRIOR ART FIG. 1b indicate a phase relation near 3-3.5 radians, which means the parameters oscillate approximately in counterphase. The phase relationship is only meaningful if the two parameters are at the same frequency. Some of the waveform pairs have no LT and phase calculations, because they were excluded for failing to meet criteria for signal wavelength matching between the two parameters.

Experimental studies in healthy subjects breathing air/CO2 mixtures, as well as clinical studies in patients with cerebrovascular pathology and head trauma, have demonstrated reduced OxyHb/deOxyHb phase values in those conditions. CO2 is believed to reduce the effectiveness of cerebrovascular regulation mechanisms. The reduced phase values in subjects following CO2 inhalation and in patients with cerebrovascular pathology or head trauma may be a result of dysregulation.

For example, PRIOR ART FIG. 2a shows LF waveforms of OxyHb and deOxyHb after bandpass filtering, and the series of lead time and phase calculations corresponding to the waveforms are shown in PRIOR ART FIG. 2b, for a hospitalized patient following a traumatic brain injury. The results shown in PRIOR ART FIG. 2b indicate a phase relation near zero radians, which means the parameters oscillate nearly in phase. Some of the waveform pairs shown in PRIOR ART FIG. 2a have no LT and phase calculations shown in the lower graph, because they were excluded for failing to meet criteria for signal wavelength matching between the two parameters.

It would therefore, be useful to provide for a device that is capable of measuring OxyHb/deOxyHb phase relationships, as a marker of cerebrovascular dysregulation. While such devices exist in the prior art, such known devices have important limitations, including:
Influence of scalp circulation was not mitigated, except by theoretical calculations of the scalp effect;
Waveform pairs of differing signal wavelengths are not excluded, confounding phase calculations;
Epochs of non-oscillatory waveforms are not excluded; and
Fourier methods were used in phase analysis, which perform poorly when signals are not strictly periodic (i.e., waveform periods are variable), and very poorly when non-stationary signals are superimposed on oscillatory signals.

As can be readily seen in the waveform comparisons mentioned above, one or another of the parameter signals may exhibit characteristics independent of the other. The causes may be related to respiration dynamics that interrupt venous outflow from the brain, for example, which may affect the measured deOxyHb disproportionately. Causes may be related to sudden change in metabolic demand resulting from mental exertion, which may affect the measured OxyHb disproportionately. In any case, the calculation of a phase between two signals of different signal wavelengths produces a meaningless result. Fourier analysis methods cannot exclude the effects such non-conforming waveforms, which reduces coherence and ultimately also

reduces sensitivity to actual physiological disturbances.

It can also be seen in the aforementioned waveform comparisons and plots that signal wavelengths do not remain constant, so the signal oscillations are not strictly periodic. Since Fourier analysis produces a phase spectrum, with discrete phase calculations at each of the spectral lines, it requires a significantly longer dataset for analysis in order obtain statistically significant results for each spectral line than would be the case for truly periodic signals.

### SUMMARY

Given the shortcomings described above, it is an aim of the present disclosure to describe a novel device that incorporates an NIRS system for measuring OxyHb/deOxyHb phase relationships that is free of the limitations of those described in the prior art. This disclosure describes embodiments of such a device, its operation, and method of use, whereby the device measures the phase relationship of two signals, representing physiological parameters such as cerebral OxyHb and deOxyHb, or any other waveform pair, in which both originate from a common quasi-periodic physiological input. Further, if the two signals represent cerebral OxyHb and deOxyHb, the device may include mechanical optical clearance (MOC) to reduce effects of scalp tissue circulation on the measurements.

Embodiments described herein are directed to a noninvasive device for evaluation of the relationship between two physiological parameters with time-varying magnitudes.

The device includes one or more biological sensors to measure a time series of values for each parameter; waveforms representing the time-varying magnitudes are identified in the time series of the first parameter; and an assessment is made of the presence and temporal relationships of corresponding waveforms in the second parameter measurement.

In at least one embodiment, the device is designed, with the use of signal bandpass filtering, to measure time-varying magnitude changes occurring only over relatively long intervals, such as signal wavelengths of 15-120 seconds, and/or only over relatively shorter time intervals, such as 2-7 seconds, in order to observe physiological effects occurring within a certain range of time scales. In some embodiments, the observed time-varying magnitude changes appear as waveforms when represented graphically along a timeline, whose signal wavelengths are within the range of time intervals defined by the signal bandpass filtering.

In some embodiments, an indication of effectiveness of cerebrovascular regulation is measured with the device by calculation of a series of time delays, corresponding to a series of waveform pairs of the two parameters; and/or by a series of phase delays between the waveform pairs, in which each time delay is normalized by the contemporaneous signal wavelength in one parameter signal and scaled to units of radians or degrees.

In at least one embodiment, the device incorporates a mechanism to calculate the two individual parameter signal wavelengths in each successive pair, and to include only those signal wavelength pairs that meet certain criteria in time delay and phase calculations. In some embodiments, a criterion will be that one parameter signal wavelength must be within 20% and/or another desirable ratio, of the other parameter signal wavelength. In some embodiments a criterion will be that one or both parameter signal wavelengths must fall within a specified range to be included; the specified range may be between 5 and 15 seconds for example.

In at least one embodiment, the device incorporates a mechanism for reducing the influence of scalp tissue on cerebral measurements. For example, in some embodiments, mechanical optical clearance (MOC) is implemented to clear blood and other fluids from the region of measurement. MOC is performed by applying regional pressure to the scalp, such as by firm contact of a sensor patch over the region of measurement. In some embodiments, MOC is performed by outwardly projecting features on the sensor elements to indent the scalp, such as through the use of optically clear lenses over the emitters and detectors in the NIRS system.

These and other embodiments are shown in the accompanying figures and escribed in greater detail below.

### BRIEF DESCRIPTION OF THE DRAWINGS

PRIOR ART FIG. 1a is a waveform graph showing LF waveforms of OxyHb and deOxyHb after bandpass filtering for a healthy individual.

PRIOR ART FIG. 1b is a graph showing a series of lead time and phase calculations corresponding to the waveforms shown in PRIOR ART FIG. 1a.

PRIOR ART FIG. 2a is a waveform graph showing LF waveforms of OxyHb and deOxyHb after bandpass filtering for a hospitalized patient following a traumatic brain injury.

PRIOR ART FIG. 2b is a graph showing a series of lead time and phase calculations corresponding to the waveforms shown in PRIOR ART FIG. 2a.

FIG. 3a is a front perspective view of an embodiment of a device for evaluation of the relationship between two physiological parameters with time-varying magnitudes, which utilizes a sensor patch.

FIG. 3b is a side perspective view of the device shown in FIG. 3a.

FIG. 4 shows a front perspective view of the device shown in FIGS. 3a-3b positioned on the head of a patient.

FIG. 5a is a detailed perspective view of the sensor patch shown in FIGS. 3a-3b, wherein the sensor patch includes a dual optical wavelength LED light source and two optical detectors.

FIG. 5b is a detailed view of the LED light source shown in FIG. 5a, showing an embodiment wherein the light source and optical detectors are equipped with hemispherical lenses.

FIG. 6a is a detailed sectional view of the interface of the sensor patch with the scalp, skull and cerebral cortex of the patient shown in FIG. 4, wherein the optical path between the light source and optical detectors is shown without the use of hemispherical lenses.

FIG. 6b is a detailed sectional view of the interface of the sensor patch with the scalp, skull and cerebral cortex of the patient shown in FIG. 4, wherein the optical path between the light source and optical detectors is shown in an embodiment where the light source and optical detectors are equipped with hemispherical lenses.

FIG. 7 is a block diagram showing the relationship of the various electronic components of the device embodiments shown in FIGS. 3a-6b.

### DETAILED DESCRIPTION

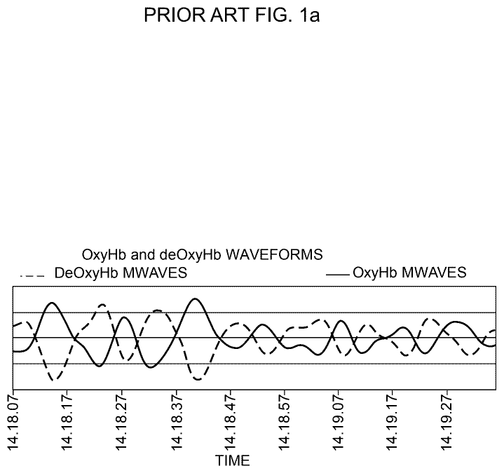

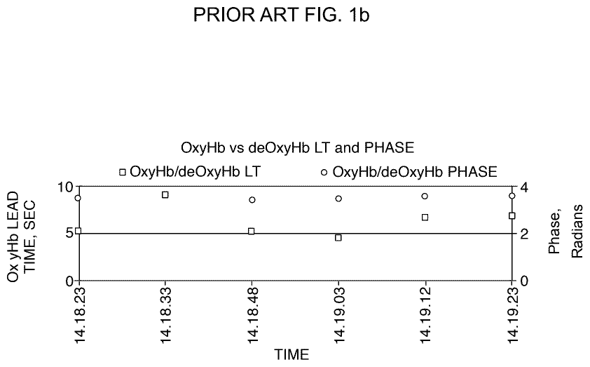

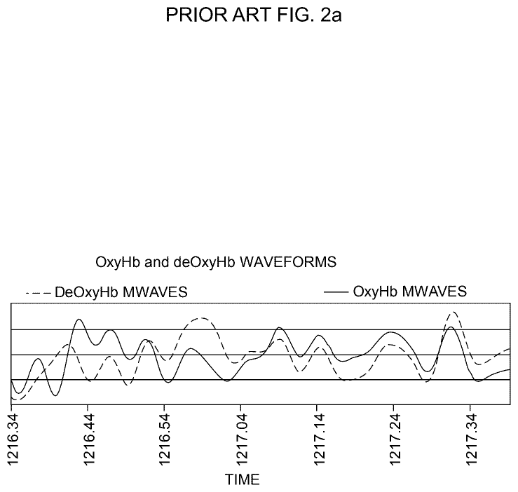

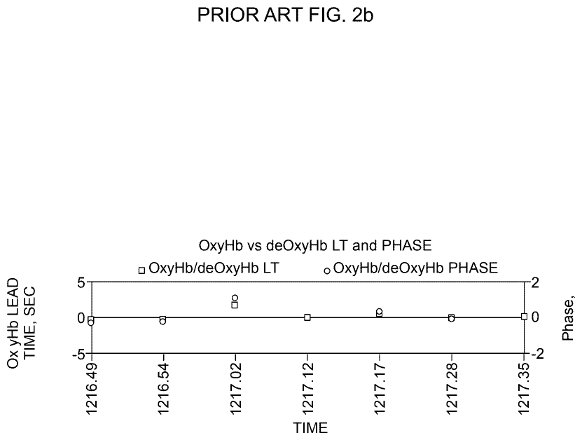

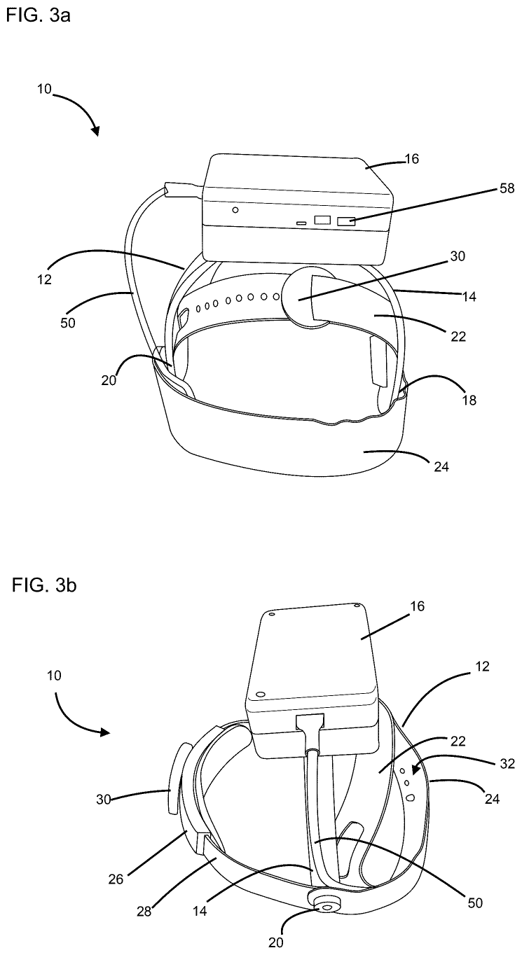

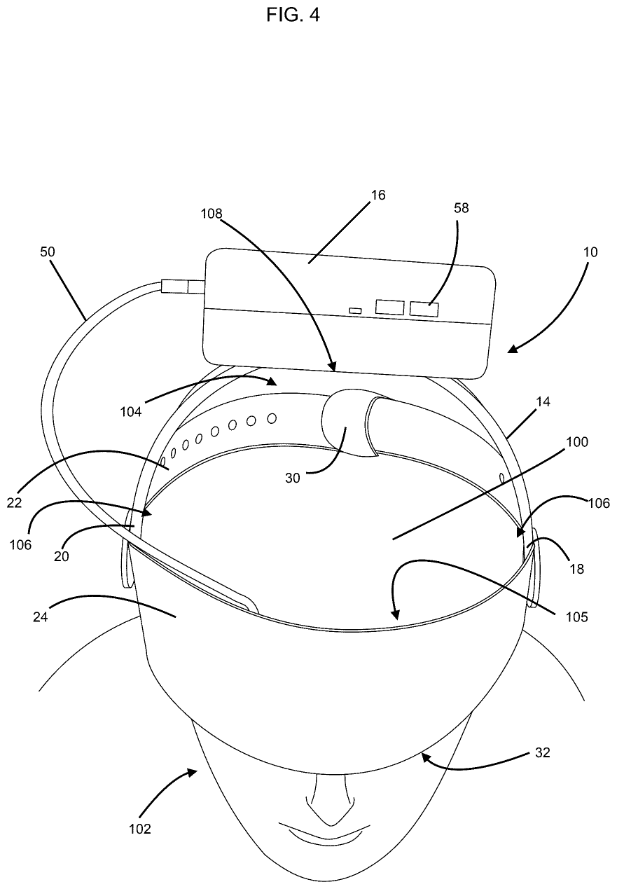

Embodiments of a unique device 10, for measuring OxyHb/deOxyHb phase relationships are shown in the accompanying drawings of FIGS. 3a-6b and described in detail below. In at least one embodiment, the device 10 includes a patient wearable headset assembly 12, configured to be worn over the head 100 of a patient 102 (see FIG. 4) when the device 10 is in use.

In at least one embodiment, the headset assembly 12 includes an electronics support member 14 which is configured to extend over the top 104 of the head 100 of the patient 102 and support an electronics assembly housing 16. Each end 18 and 20 (see FIGS. 3a and 4) of the electronics support member 14 is engaged to an adjustable circumferential member 28 and a sensor patch 24, the latter of which is typically positioned across the forehead 105 of the patient 102 when the device is in use.

In the embodiment shown in FIG. 3b, the headset assembly 12 is shown to include an adjustable circumferential member 28 which is configured to extend circumferentially away from the sensor patch 24, around the sides 106 and back 108 of the head 100. The member 28 includes an adjustable tensioning mechanism 30, which can be a tensioning knob, buckle, or other conventional adjustment mechanism in order to loosen or tighten the fit of the headset assembly around the patient's head 100. The adjustable frame strap 22 may be loosened or tightened across the patient's head 100 via an adjustment buckle or other conventional adjustment mechanism 30.

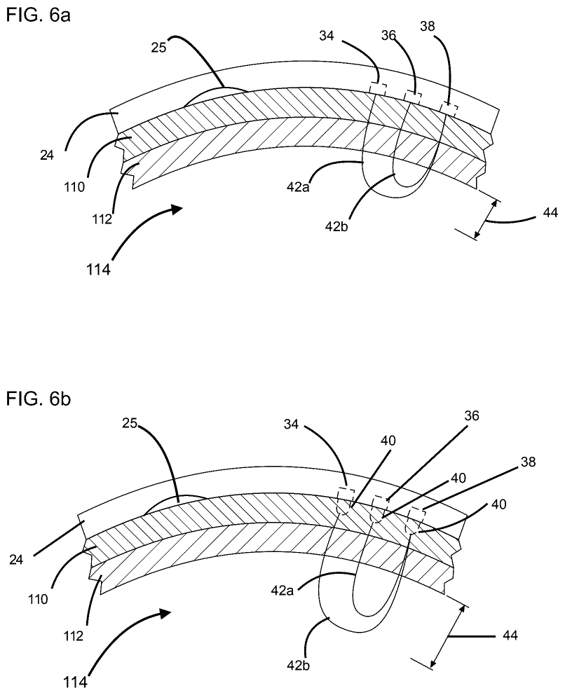

The various supports and adjustment mechanisms of the headset assembly 12 help to ensure that the operational surface 32 of the sensor patch 24 is properly positioned and held in place against the forehead 105 and scalp 110 (see FIGS. 6a and 6b) of the patient's head 100 during use of the device 10 to optimize MOC by compression of the region generally under the patch 24.

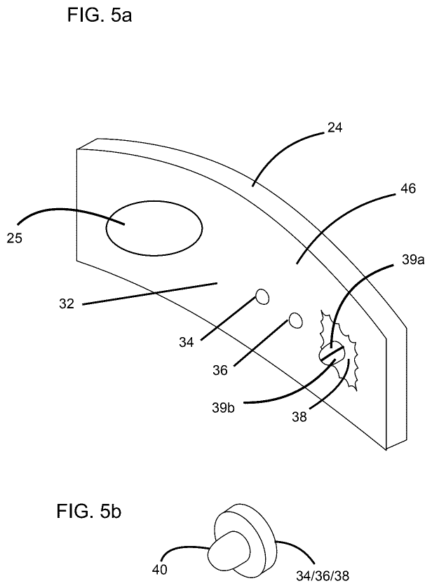

As is shown in the embodiment of FIG. 5a, the operational surface 32 of the sensor patch 24 includes at least two light sources 39a and 39b which, in at least one embodiment, is a single light source 38 capable of transmitting light at two or more different optical wavelengths. In at least one embodiment, the light source 38 is a pair of light emitting diodes (LEDs), each of which emits a different optical wavelength. In the embodiment shown, the operational surface 32 of the sensor patch 24 also includes at least two optical detectors 34 and 36, which provide for two or more optical pathlengths by virtue of their differing separation distances from the light source 38.

In at least one embodiment, the two or more optical wavelengths emitted by the light source 38 are respectively optimized for sensitivity to OxyHb and deOxyHb.

In at least one embodiment, such as is shown in FIG. 5b, lenses 40 may be attached to the light source 38 and detectors 34 and 36, which serve to optimize optical performance and provide MOC by local displacement of scalp tissue out of the optical pathways 42a and 42b such as in the manner illustrated in FIG. 6b. The lenses 40 may be hemispherical, or approximately hemispherical, such as may be obtained by a suitable amount of optically clear epoxy deposited and cured on the transparent cover of the component. Other lens configurations may be used, such as aspheric or convex lenses.

The performance benefit provided to the device 10 through the use of lenses 40 on the light source 38, and detectors 34 and 36 is made apparent by comparing the characteristics of an embodiment of the device 10 without lenses 40, such as is shown in FIG. 6a, compared to an embodiment of the device 10 with lenses 40 equipped such as is shown ion FIG. 6b.

As shown, when the device 10 is in use, optical pathways 42a and 42b extend from the light source 38, through the patient's scalp 110, skull 112 and into the cerebral cortex 114, eventually reaching one of the detectors 34 and 36. As first detector 34 is displaced a greater distance away from the light source 38 as compared to the position of second detector 36 the respective optical pathways 42a and 42b between the light source 38 and each detector 34 and 36 will have a different length. The use of lenses 40 on the light source 38, and detectors 34 and 36, such as in the manner shown in FIG. 6b provides optical pathways 42a and 42b which have a greater measurement depth 44 into the cerebral cortex than an embodiment of the device 10 that lacks such lenses would be capable of, such as is illustrated in FIG. 6a.

In at least one embodiment, an example of which is shown in FIG. 5a, the operational surface 32 of the sensor patch 24 that surrounds the optical components (i.e., the light sources 38, and detectors 34 and 36) is made up of optically opaque material 46, which blocks ambient light from contaminating the optical signal received by the detectors 34 and 36 from the light source 38.

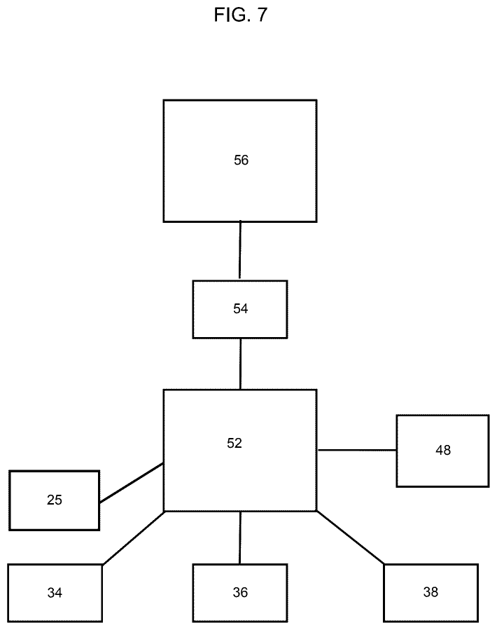

In the various embodiments shown, the optical elements 34, 36, and 38 are in electronic communication with a power supply 48 (shown in FIG. 7) contained within the housing 16 via wire 50.

The power supply 48 is in electronic communication with a controller 52. Controller 52 controls the electric power provided to the optical elements 34, 36, and 38. These may be cycled on and off such that only one LED optical wavelength is emitted and detected at any time, at cycling rates sufficient to permit measurements of physiological changes occurring on fast timescales. A desirable timescale may be 50 milliseconds for each complete measurement, with individual LED “on” times required to be approximately the measurement timescale multiplied by 1/N where N is the number of LED optical wavelengths.

In at least one embodiment, the controller 52 is a microprocessor, that measures and digitizes the photocurrent signals received from the optical detectors 34 and 36 after the optical signals transmitted by the light sources 38 have passed into the cerebral cortex 114 and been detected by the at least one optical detectors 34 and 36 in the manner illustrated by optical pathways 42a and 42b shown in the embodiments of FIGS. 6a and 6b.

In addition to the above, the controller 52 performs the following functions:
a) Calculate the OxyHb and deOxyHb from the digitized signals for each time point. Time points may be 50 milliseconds apart.
b) Remove the higher frequency components in the calculated OxyHb and deOxyHb time series, including cardiac waveforms and other signal frequencies above a selected frequency range. In at least one embodiment, this is accomplished by digital lowpass filtering, or by signal averaging, for example. A selected frequency range may be 5 -15 seconds signal wavelength (0.067 to 0.2 Hz).
c) Remove the low frequency non-periodic and periodic signal components below the selected frequency range. In at least one embodiment, this is accomplished by digital highpass filtering, or by subtracting a moving average trendline from the lowpass filtered signal, for example.
d) Analyze the signal waveforms to determine, for each signal waveform, whether there is a valid OxyHb/deOxyHb signal waveform pair that meets selected criteria. In at least one embodiment, the criteria includes signal wavelength equality (i.e., signal waveforms are of approximately equal length), signal wavelength within limits (i.e., to exclude signal waveforms that may be misformed, or too long or too short to qualify as LF), or other criterion.
e) Analyze the valid signal waveform pairs to determine the OxyHb/deOxyHb phase of each pair, based on the lead time of one parameter versus the other and the signal wavelength for that pair.
f) Calculate an average phase value from a sequence of phase values determined by the analysis. In at least one embodiment, the average phase value is an average over a complete dataset, or a running average that is updated periodically with the most recent values during continuous measurement. In at least one embodiment, the average phase value is described as a non-parametric “Index” or as a phase in units of radians or degrees.

In at least one embodiment, the housing 16 contains a radio transmitter and receiver such as a Bluetooth wireless communication module 54 in communication with the controller 52. Through a Bluetooth connection, the controller 52 is in communication with a laptop or other device having a display 56 for graphically presenting the results of the average phase value and/or index calculations. The display 56 may also present a graphical representation of phase values along a timeline such as in the manner of the graphs shown in PRIOR ART FIGS. 1a-2b.

In at least one embodiment, the controller 52 and/or the display 56 includes an alarm mechanism (not shown). The alarm mechanism may be any sort of conventional alarm mechanism that provides an audible or visual indicator when triggered. A physician or other individual who is administering use of the device 10 to a patient may want to be notified that one or more threshold phase values among those being calculated by the controller 52 has been determined. When such a threshold value is determined to by present by the controller 52, the alarm will be triggered and the physician notified.

In at least one embodiment, an example of which is shown in FIG. 5a the operational surface 32 of the sensor patch 24 includes a pressure sensor 25 for detecting continuous arterial blood pressure (AP). The pressure sensor 25 communicates AP measurements to the controller 52 (and display 56) during use of the device 10.

In at least one embodiment, AP measurements received by the controller 52 are converted into digitized values over a series of time points to generate a continuous time-varying AP signal. The AP signal is processed and filtered by the controller in the same manner as the OxyHb signal or deOxyHb signal described above. For example, the controller 52 is configured to remove higher frequency components in the calculated AP signal above a selected LF range and/or remove low frequency non-periodic and periodic components from the calculated AP signal below the selected LF range. In at least one embodiment, the controller 52 is configured to analyze the AP signal to determine whether there is a valid AP/OxyHb waveform pair that meets selected criteria, then analyze any valid waveform pairs to determine an AP/OxyHb phase of each pair. The controller 52 then calculates an average phase value result from a sequence of phase values. These time series of calculated phase value results are then transmitted by the controller 52 to the display 56 which graphically presents the series of phase value calculations and the average result.

In at least one embodiment, the device 10 includes one or more direct wire ports 58 (visible in FIGS. 3a and 4) suitable to provide a connection for direct wired communication between the controller 52 and display 56. In some embodiments, the housing 16 includes ports configured to provide a wired connection for supplying electrical power to the power supply 48. In some embodiments, the power supply 48 includes a battery (not shown).

The many features and advantages of the invention are apparent from the above description, chiefly the serial real-time phase measurements on individual signal waveforms, the reduced effect of scalp tissue by MOC, and the exclusion of non-paired parameter signal waveforms from analysis. Numerous modifications and variations will readily occur to those skilled in the art. For example, other parameter pairs may be analyzed, such as arterial pressure (AP) and OxyHb, AP and cerebral blood flow (CBF), AP and total Hb, or CBF and OxyHb. Since such modifications are possible with different sensors, the invention is not to be limited to the exact construction and operation illustrated and described. Rather, the present invention should be limited only by the following claims.

What is claimed is:

1. A device for measuring OxyHb/deOxyHb phase relationships, the device comprising:
a patient wearable headset assembly, the patient wearable headset assembly having a sensor patch, the sensor patch having an operational surface configured to be placed against a patient's scalp when the patient wearable headset assembly is placed over a patient's head,
the sensor patch comprises at least one light source and at least two optical detectors, the at least two optical detectors are separated from the at least one light source by different distances so as to create a first optical pathway and a second optical pathway, wherein the first optical pathway has a first length and the second optical pathway has a second length, the first length being different from the second length, the at least one light source is constructed and arranged to emit light of a first optical wavelength that is transmitted through an area of scalp, skull and into an area of a patient's cerebral cortex, and light of a second optical wavelength that is transmitted through the area of scalp and the skull and into the cerebral cortex, the light of the first optical wavelength having different wavelength characteristics from that of the light of the second optical wavelength, wherein the different optical wavelength characteristics combined with the different optical pathway lengths determine OxyHb and deOxyHb concentrations;
the patient wearable headset assembly further comprises a controller, the controller in electronic communication with the light source, the at least two optical detectors, and a display, the controller configured for:
a) measuring and digitizing the light of the first optical wavelength and the light of the second optical wavelength detected by the at least two optical detectors into digitized values;
b) calculating OxyHb and deOxyHb concentrations from the digitized values over a series of time points to generate continuous time-varying OxyHb and deOxyHb signals;
c) removing higher frequency components in the generated continuous time-varying OxyHb and deOxyHb signals above a selected frequency range;
d) removing low frequency non-periodic and periodic signal components in the generated continuous time-varying OxyHb and deOxyHb signals below the selected frequency range;
e) analyzing each of the generated continuous time-varying OxyHb and deOxyHb signals to determine whether there is a valid OxyHb/deOxyHb signal waveform pair that meets selected criteria;
f) analyzing any valid signal waveform pairs to determine an OxyHb/deOxyHb phase of each pair;
g) calculating an average phase value result from a sequence of phase values; and
h) transmitting a time series of calculated phase value results to the display, the display configured to graphically present the time series of calculated phase value results and an average result.

2. The device of claim 1, wherein the patient wearable headset assembly further comprises:
an adjustable circumferential member that is engaged to the sensor patch and is configured to extend around a back of the patient's head when the patient wearable headset assembly is placed over the patient's head, and is further configured to permit tensioning of the sensor patch and thereby apply pressure to the scalp.

3. The device of claim 2, wherein the patient wearable headset assembly further comprises:
an electronics support member, the electronics support member extending from a first side of the sensor patch to a second side of the sensor patch, the electronics support member configured to extend over the top of the patient's head when the patient wearable headset assembly is placed over the patient's head; and
an electronics assembly housing, the electronics assembly housing is engaged to and supported by the electronics support member, the electronics assembly housing containing the controller.

4. The device of claim 3, further comprising a power supply, the power supply being contained within the electronics assembly housing, the power supply being in electronic communication with the controller.

5. The device of claim 4, further comprising a Bluetooth module, the Bluetooth module being contained within the electronics assembly housing, the Bluetooth module configured to provide wireless communication between the controller and the display.

6. The device of claim 1, wherein the at least one light source and the at least two optical detectors are each fitted with an optical lens.

7. The device of claim 6, wherein the optical lens is hemi-spherical in shape.

8. The device of claim 1, wherein the series of time points are 50 milliseconds apart.

9. The device of claim 1, wherein the removal of the higher frequency components in the generated continuous time-varying OxyHb and deOxyHb signals above the selected frequency range is accomplished by digital lowpass filtering.

10. The device of claim 1, wherein the selected frequency range is 5 -15 seconds signal wavelength (0.067 to 0.2 Hz).

11. The device of claim 1, wherein the selected frequency range is 15-120 seconds.

12. The device of claim 1, wherein the selected frequency range is 2-7 seconds.

13. The device of claim 1, wherein the removal of the low frequency non-periodic and periodic signal components below the selected frequency range is accomplished by digital highpass filtering.

14. The device of claim 1, wherein the selected criteria include at least one member of the group consisting of: signal wavelength equality and signal wavelength within limits.

15. The device of claim 1, wherein the time series of calculated phase value results is analyzed to provide a graphical presentation of OxyHb/deOxyHb phase values versus signal wavelength.

16. The device of claim 1, wherein the average phase value result is a running average that is updated periodically during continuous measurement.

17. The device of claim 1, wherein the average phase value result is a non-parametric index.

18. The device of claim 1, wherein the calculated average phase value result includes a threshold phase value, the controller including an alarm function, the alarm being activated when the threshold phase value is reached.

19. The device of claim 1, wherein the operational surface of the sensor patch includes a pressure sensor.

20. A patient wearable Near Infrared Spectroscopy (NIRS) device for measuring phase relationships, the device comprising:
a patient wearable headset assembly, the patient wearable headset assembly having a sensor patch, the sensor patch having an operational surface configured to be placed against a patient's scalp when the patient wearable headset assembly is placed over a patient's head, the operational surface of the sensor patch configured to include a pressure sensor to measure continuous arterial blood pressure (AP),
the sensor patch comprises at least one light source and at least two optical detectors, the at least two optical detectors are separated from the at least one light source by different distances so as to create a first optical pathway and a second optical pathway, wherein the first optical pathway has a first length and the second optical pathway has a second length, the first length being different from the second length, the at least one light source is constructed and arranged to emit light of a first optical wavelength that is transmitted through an area of scalp, skull and into an area of a patient's cerebral cortex, and light of a second optical wavelength that is transmitted through the area of scalp and the skull and into the cerebral cortex, the light of the first optical wavelength having different wavelength characteristics from that of the light of the second optical wavelength, wherein the different optical wavelength characteristics combined with the different optical pathway lengths determine OxyHb concentrations;
the patient wearable headset assembly further comprises a controller, the controller in electronic communication with the operational surface, the at least one light source, the at least two optical detectors, and a display, the controller configured for:
a) measuring and digitizing the light of the first optical wavelength and the light of the second optical wavelength detected by the at least two optical detectors into digitized values;
b) calculating OxyHb concentrations from the digitized values over a series of time points to generate a continuous time-varying OxyHb signal;
c) measuring and digitizing an electrical signal from the pressure sensor to produce digitized AP values over a series of time points to generate a continuous AP time-varying signal;
d) removing higher frequency components in the generated continuous time-varying OxyHb signal and the continuous AP time-varying signal above a selected frequency range;
e) removing low frequency non-periodic and periodic signal components in the generated continuous time-varying OxyHb signal and the the continuous AP time-varying signal below the selected frequency range;
f) analyzing the generated continuous time-varying OxyHb signal and the continuous AP time-varying signal to determine whether there is a valid AP/OxyHb signal waveform pair that meets selected criteria;
g) analyzing any valid signal waveform pairs to determine an AP/OxyHb phase of each pair;
h) calculating an average phase value result from a sequence of phase values; and
i) transmitting a time series of calculated phase value results to the display, the display configured to graphically present the time series of calculated phase value results and an average result.
